(19) United States
(12) Patent Application Publication
(10) Pub. No.: US 2010/0249716 A1
(43) Pub. Date: Sep. 30, 2010

(54) Single-Use Ampoule

(75) Inventor: Lance Wong, San Mateo, CA (US)

Correspondence Address: THE H.T. THAN LAW GROUP, WATERFRONT CENTER SUITE 560, 1010 WISCONSIN AVENUE NW, WASHINGTON, DC, 20007, US

(73) Assignee: MEDIMMUNE VACCINES, INC., Gaithersburg, MD (US)

(21) Appl. No.: 12/715,041

(22) Filed: Mar. 1, 2010

Related U.S. Application Data
(62) Division of application No. 11/864,361, filed on Sep. 28, 2007, now abandoned.

(51) Int. Cl. A61J 1/14; A61M 31/00

(52) U.S. Cl. 604/181; 604/403

(57) ABSTRACT

A single-use, blow-molded container for medical or pharmaceutical media is presented. The container comprises a flexible storage portion connected to one end a dispensing portion and a cap connected to the other end of the dispensing portion, wherein the cap is separated from the dispensing portion to create an outlet opening so that the medical media can be expelled from the flexible storage portion. One improvement comprises at least one barrier member disposed on the dispensing portion, wherein the barrier member is sized and dimensioned to limit the insertion of the dispensing member into a patient's nostril. Another improvement comprises an orifice controlling the pattern and direction of the medical media as it exits the ampoule container and wherein the orifice is spaced apart from the outlet opening.

### CROSS REFERENCE TO RELATED APPLICATION

This application is a divisional application of U.S. patent application Ser. No. 11/864,361, filed on Sep. 28, 2007, which is incorporated herein by reference in its entirety.

### FIELD OF THE INVENTION

The present invention generally relates to hermetically-sealed containers for all types of media and particularly to aseptic ampoules for the delivery of predetermined doses of medical media and other liquids.

### BACKGROUND OF THE INVENTION

Containers for the delivery of measured doses of medical liquids are well known in the art. For instance, U.S. Pat. No. 3,917,120 discloses a blow-molded container comprising a main chamber for housing a pharmaceutical medium, a closure portion and a narrower measuring chamber located between the main chamber and the closure portion having an aperture on its tip, through which the pharmaceutical medium may be dispensed. The container may further comprise sheet-like extensions of material disposed on the sides of the main chamber that may serve as bridging members to join individual containers into connected blocks of containers. The bridging members may include a weakened tear line running in the longitudinal direction for easier separation of individual containers from the connected block of containers.

Another type of container for medical media is disclosed in U.S. Pat. No. 5,046,627, wherein an ampoule comprises a main chamber for containing medical media, a cap, and a neck portion located between the main chamber and the cap, said neck portion having a number of differently sized segments and being configured to receive the conical member located on one end of a hypodermic syringe. The cap of said ampoule is connected to the neck at breaking point having a reduced wall thickness. The cap may additionally have a tip-stretched toggle to aid in grasping the ampoule. As in the '120 patent, the ampoule of the '627 patent is constructed by blow-molding.

U.S. Pat. No. 4,469,254 discloses yet another container, this one comprising a body portion for containing a pharmaceutical medium and a dosing chamber connected to said body portion. The diameter of the dosing chamber increases in a stepwise fashion, the portion of the dosing chamber connecting to the body portion having the largest diameter and the portion of the dosing chamber at its distal end having the smallest diameter. The dosing chamber may also include annular grooves at points along its length to aid in media containment. The distal end of the dosing chamber connects to a cap that is designed to break off, to create an opening at the distal end of the dosing chamber through which the medium is dispensed.

All of the above-described containers can be made by means of blow-fill-seal technology to ensure the aseptic quality of their contents. This technique allows containers to be constructed, filled with media and sealed in one continuous process and in one sterile environment. Unlike other packaging processes, which require separate parts to be arranged and connected and which necessitate the movement of parts among various machines, blow-fill-seal technology can take place within one sterile machine and without human intervention, vastly reducing the possibility of contamination of the container or its contents. The blow-fill-seal process is particularly valuable in the production of containers for medical or pharmaceutical media, as the sterility of the package and its contents is desirable.

Though a number of blow-fill-seal containers for pharmaceutical products exist, there remains a need in the art for containers that allow users to easily and efficiently open the package and to administer the contents in a precise fashion. Further, their remains a need in the art for containers that include mechanisms to optimize the comfort and safety of the user.

### SUMMARY OF THE INVENTION

The present invention is directed to a blow-molded container for the storage and administration of medical or pharmaceutical media. More particularly, the present invention is directed to an ampoule manufactured according to the blow-fill-seal technique for the containment of medical or pharmaceutical media and the administration of said media to the nasal passageways.

The ampoule of the present invention generally comprises improvements to a conventional hollow single use ampoule for the containment of medical media. One improvement comprises two barrier members disposed to the sides of a dispensing member of the single use ampoule. The barrier members create a physical obstruction against the insertion of the dispensing member into the nostril at the point of the barrier members, thereby preventing the user from inserting the ampoule too far into the nostril and causing discomfort or damage to the nasal passage.

Another improvement is separating the line where a cap is separated from the dispensing member or section from the point where a spray pattern is formed when the medical media is expelled. Hence, the spay pattern is not affected by the tear line when the cap is removed. The distal or exit end of the single use ampoule defines an hour-glass shape with a wide proximal end, a narrowed waist and a wide distal end, wherein the wide distal end defines the opening of the dispensing member subsequent to the separation of the closure member from the dispensing member, and the narrowed waist defines the spray pattern of the exiting medical media.

### BRIEF DESCRIPTION OF THE DRAWINGS

FIG. 1 is a front plan view of an ampoule according to the present invention;

FIGS. 2 and 3 are a perspective view and a side plan view, respectively, of the ampoule of FIG. 1;

FIG. 4 is a front plan view of a number of connected ampoules according to an embodiment of the present invention;

FIG. 5 is a top plan view of the connected ampoules of FIG. 4;

FIG. 6 is a front plan view of the ampoule of FIG. 1 with the closure portion removed;

FIG. 7 is a perspective view of the ampoule of FIG. 6;

FIG. 8 is an enlarged front plan view of a portion of the storage member, the dispensing member, the closure member and the tab of the ampoule of FIG. 1; and

FIG. 9 shows the ampoule of FIG. 8, however having a modified dispensing member.

### DETAILED DESCRIPTION OF THE INVENTION

The present invention is directed to a blow-fill-seal container for medical media and particularly for medical media that is intended for dispersement directly into the nasal passages. Further, the present invention is directed to a single-use blow-fill-seal container.

Referring to the drawings, ampoule 10 of the present invention generally comprises storage member 12, closure member 16, dispensing member 14 connecting storage member 12 to closure member 16, opening 20, through which media is dispensed, and end 32. Ampoule 10 further comprises tab 24 extending from closure portion 16 and tab 26 extending from end 32. Ampoule 10 comprises a moldable plastic material, such as polyamide or polyolefin or other materials suitable for blow-fill-seal manufacturing.

Storage member 12 of ampoule 10 is a hollow, blow-molded member for containing medical or pharmaceutical media, including liquids, gels and other media. Storage member 12 preferably has a storage capacity sufficient to hold an adequate dosage of medicines, vaccines or other liquids, and comprises a collapsible material, so that the contents of said storage member may be pushed or ejected through dispensing member 14 and opening 20 upon application of a squeezing force on storage member 12. Dispensing member 14 connects to storage member 12 and is a hollow body having a tapered, tubular shape, having a first diameter at the end connecting to storage member 12 and a second diameter at the end connecting the closure member 16, wherein the first diameter can be larger than the second diameter. The hollow tubular shape of dispensing member 14 provides a channel through which media pass when pressure is applied to the walls of storage member 12. The average diameter of dispensing member 14 is preferably smaller than the average diameter of storage member 12 to facilitate the dispersement of media in the form of a stream or a series of drops. The diameter of dispensing member 14 and opening 20 may depend upon the viscosity of the media contained in the ampoule.

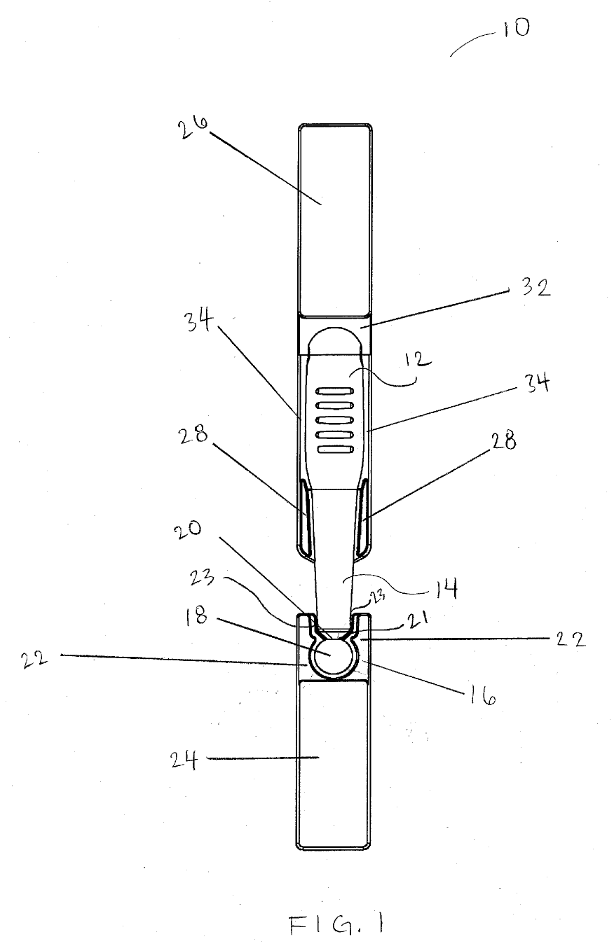

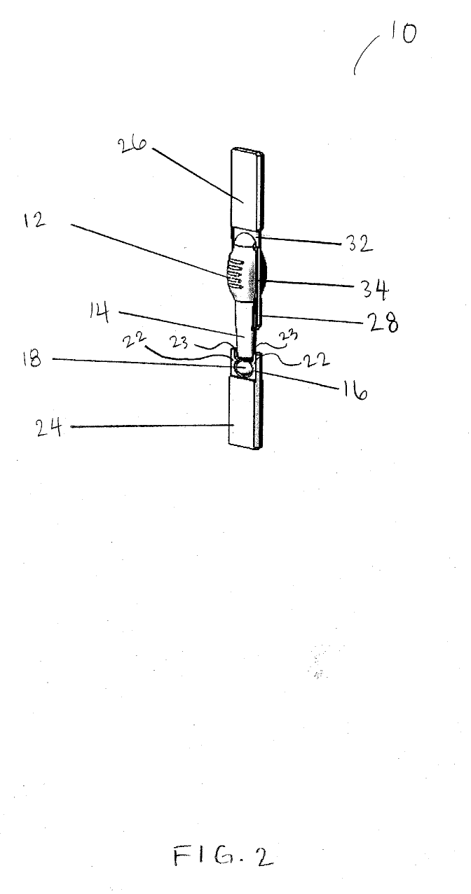

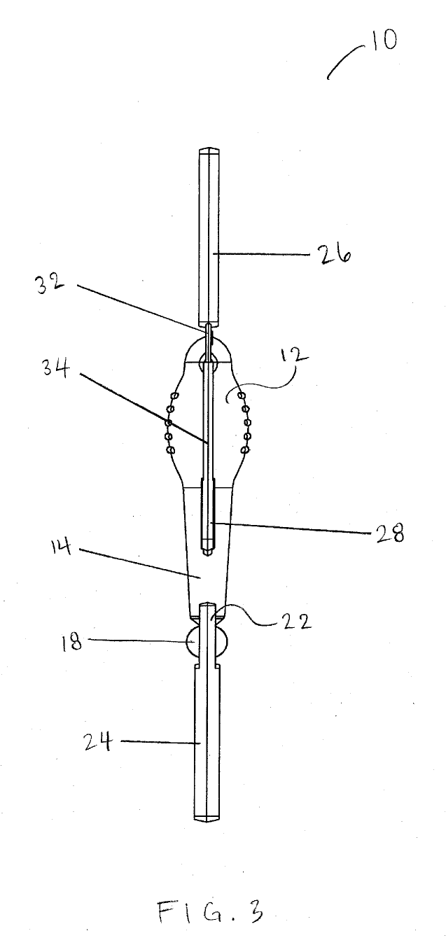

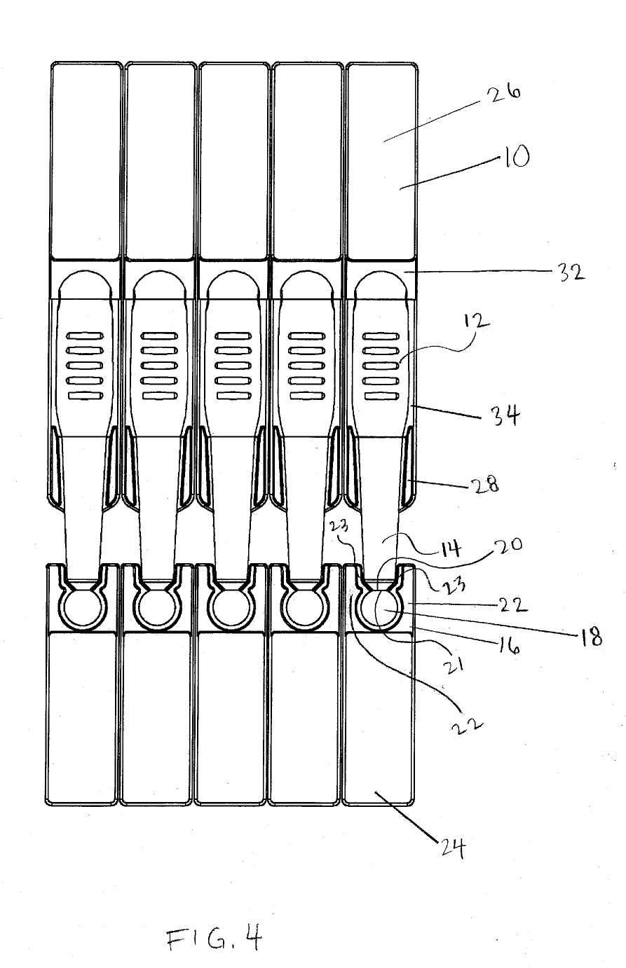

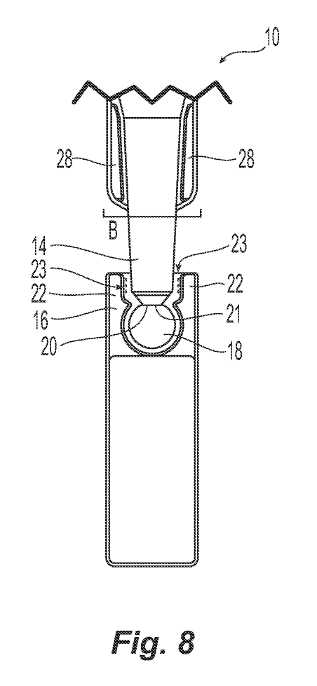

To prevent the ampoule from discarding its contents unintentionally, closure member 16 is disposed to the distal end of dispensing member 14 at opening 20. Closure member 16, best shown in FIGS. 1 and 4, comprises ball 18 and reinforcement sheets 22. Ball 18 is a hollow spherical body that connects to dispensing member 14 at separation line 21. Reinforcement sheets 22 are disposed to the sides of ball 18, extend past separation line 21 and are disposed to a portion of the sides of the distal end of dispensing member 14, as shown in FIGS. 1-4. Reinforcement sheets 22 meet dispensing member 14 at weakened line 23, which comprises the same material as other members of ampoule 10 however having a reduced thickness than the surrounding material, or having a scored texture. The inclusion of weakened line 23, shown in detail in FIG. 8, allows a user to separate reinforcement sheets 22 from dispensing member 14 by applying a twisting force to closure member 16. Reinforcement sheets 22 provide a gripping surface to facilitate the twisting motion and also provide reinforcement of the seal created by closure member 16 around opening 20.

A tab 24 of material in the form of a sheet-like body extends off of the distal end of closure member 16. Tab 24 is preferably rectangular, being longer in the longitudinal direction than in the latitudinal direction. The wider, flat sides of tab 24 may be printed with indicia or other markings. Tab 24 provides an extra gripping surface for holding ampoule 10 or applying the twisting motion necessary to separate closure member 16 from dispensing member 14. In accordance with this invention, the longitudinal sides of tab 24 are flush with the longitudinal sides of reinforcement sheets 22.

On the opposite or proximal end of ampoule 10, a tab 26 of material in the form of a sheet-like body extends from end 32 to provide yet another gripping surface for holding ampoule 10 or applying the twisting force. Like tab 24, tab 26 has a rectangular shape and is longer in the longitudinal direction than in the latitudinal direction. Indicia or other markings may be printed on the flat, wider sides of tab 26. The longitudinal sides of tab 26 are preferably flush with the longitudinal sides of end 32. Additionally, the longitudinal sides of tab 26 are flush with sides 34 and the longitudinal sides of reinforcement sheets 22 and tab 24.

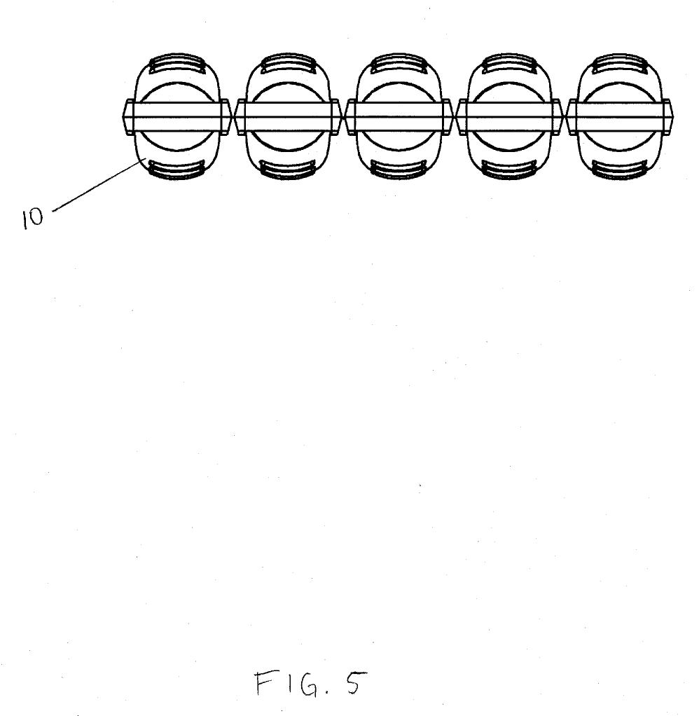

Ampoules of the present invention may be manufactured in blocks of several connected ampoules, as shown in FIGS. 4 and 5. According to the present invention, ampoules connect, via a strip of material having a reduced thickness than the surrounding material, at the longitudinal sides of tab 26, end 32, sides 34, closure member 16 and tab 24. An individual ampoule may be separated from a block of connected ampoules by applying enough force at the longitudinal sides of said ampoule to breach the material connection between it and the ampoules connected to it.

The ampoule described above is shown in the parent '032 patent application. However, the improvements described below can be used with any conventional single use ampoules. Non-limiting examples of suitable ampoules include, but are not limited to, U.S. Pat. Nos. 5,577,636; 3,917,120; 5,482,193; 4,469,254; D259,213, 5,009,309; D330,160; D362,304 and D309,097, among others.

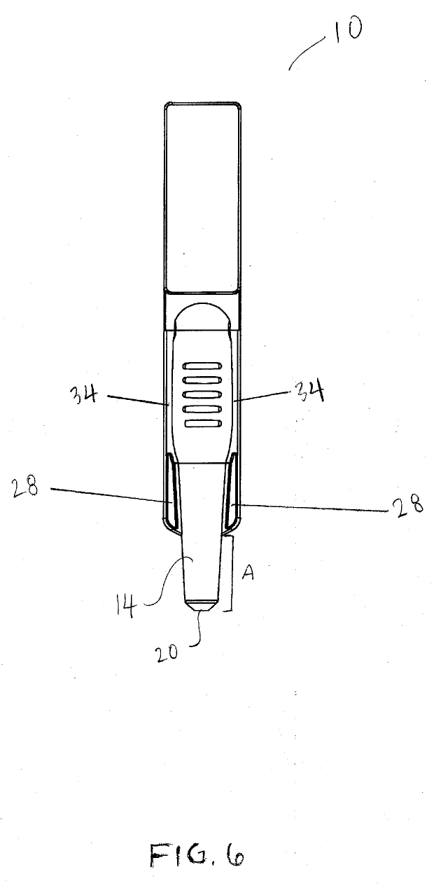

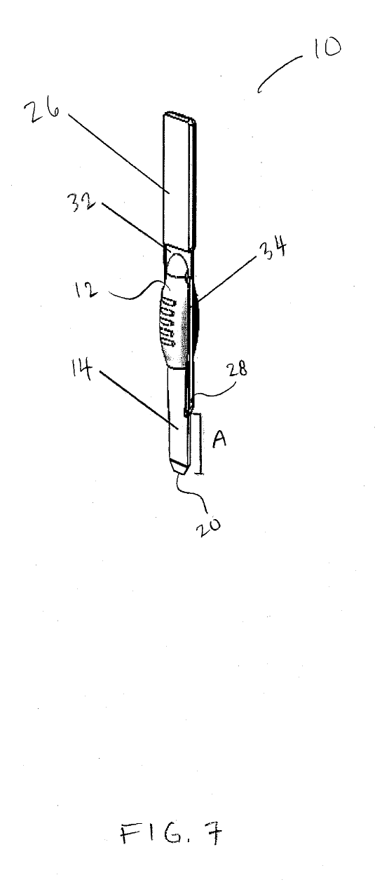

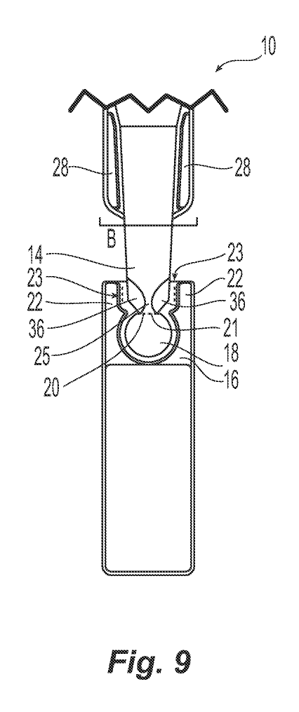

The ampoule of the present invention is preferably used in the administration of medical liquids in the nasal passages. One improvement of the present invention includes barriers 28 disposed to the sides of dispensing member 14, effectively widening the ampoule with respect to the width of dispensing member 14. As best shown in FIGS. 6 and 7, in which closure member 16 and tab 24 have been separated from the rest of ampoule 10, barriers 28 are sheet-like extensions of material that begin at the distal ends of sides 34 and extend along approximately half of the length of dispensing member 14. Barriers 28 provide a physical obstruction to the insertion of dispensing member 14 into a human nostril. A user may only insert dispensing member 14 comfortably and easily into a nostril for the purpose of dispersing the contents of ampoule 10 into the nasal passage up to the point of barriers 28. In accordance with the present invention, the distance A, shown in FIGS. 6 and 7, describes the distance between the distal end of barriers 28 and opening 20. Distance A is the distance where the spray of the medical media is most effective to enter the nasal cavity and to minimize dripping of the media outside of the nostrils. The width B, shown in FIGS. 8 and 9, describes the distance from the external edge of one barrier 28 to the external edge of opposite barrier 28. Width B is designed to be larger than the nostril's opening to create an interference with the insertion of dispensing member 14. Ampoule 10 can be made available with various distances A and widths B to match the nostril size of the recipients, e.g., infants, toddlers, teens, adults, etc.

For infants and toddlers, distance A can be in the range of about 2 mm to about 7 mm, preferably from about 3 mm to about 6 mm and more preferably from about 4 mm to about 5 mm, and width B can be in the range of about 4 mm to about 8 mm, preferably from about 5 mm to about 7 mm, and more preferably from about 5.5 mm to about 6.5 mm. For teenagers and adults, distance A can be in the range of about 5 mm to about 12 mm, preferably from about 7 mm to about 10 mm and more preferably from about 8 mm to about 9 mm, and width B can be in the range of about 5 mm to about 12 mm, preferably from about 7 mm to about 10 mm, and more preferably from about 8 mm to about 9 mm.

Ampoule 10 may be used to deliver its contents into the nasal passages in a drop-like fashion or in a spray- or stream-like fashion. Typically, a user will remove closure member 16 from dispensing member 14 to reveal opening 20 and apply a force on storage member 12 to direct a spray or stream of medical media into the nasal passage. Upon removing closure member 16 from dispensing member 14, the material surrounding opening 20 should have a level, non-jagged surface, free from any overhanging material or pieces of material that were not cleanly separated from dispensing member 14 by a twisting motion from the user. Jagged opening 20 can cause the spray pattern to be irregular and can direct the media stream or spray sideway or away from the nasal cavity. To facilitate a clean separation of the material comprising dispensing member 14 from the material comprising closure member 16, separation line 21 can be scored or pseudo-perforated, i.e., the perforations do not cut through the material.

Another improvement of the present invention includes a modified dispensing member, as shown in FIG. 9. In accordance with this embodiment, dispensing member 14 includes fillets 36, comprising flattened sections of material having rounded inner edges so as to define an orifice 25 through which media may travel. Fillets 36 create a dispensing member having an hourglass-like shape, comprising a wide proximal portion, a narrowed waist with orifice 25 and a wide distal portion. The distal portion includes opening 20 of dispensing member 14 and separation line 21. The user may expose opening 20 by separating closure member 16 from dispensing member 14 at separation line 21. Preferably, separation line 21 is scored or has pseudo-perforation to encourage a non-jagged opening 20. However, since separation line 21/opening 20 are spaced apart from orifice 25, where the spray pattern is defined, the spray pattern is not affected by the shape of opening 20. Orifice 25 is sized and dimensioned to establish the desired spray pattern depending on the viscosity and density of the medical media contained in ampoule 10. Orifice 25 should not be too small, since that can cause the exiting media to have a high velocity. Orifice 25 should not be too large, since the media can drip out of the ampoule. The diameter of orifice 25 can be in the range of about 0.2 mm to about 0.8 mm, more preferably about 0.4 mm to about 0.6 mm and more preferably about 0.45 mm to about 0.55 mm. Opening 20 and the distal portion of the hourglass shape should be sufficiently large and diverge so as not to interfere with the spray pattern from orifice 25.

While it is apparent that the illustrative embodiments of the invention disclosed herein fulfill the objectives of the present invention, it is appreciated that numerous modifications and other embodiments may be devised by those skilled in the art. Additionally, feature(s) and/or element(s) from any embodiment may be used singly or in combination with other embodiment(s) and steps or elements from methods in accordance with the present invention can be executed or performed in any suitable order. Therefore, it will be understood that the appended claims are intended to cover all such modifications and embodiments, which would come within the spirit and scope of the present invention.

1-2. (canceled)

3. A blow-molded ampoule container for medical media comprising a flexible storage portion connected to one end of a dispensing portion and a cap connected to the other end of the dispensing portion, wherein the cap is separated from the dispensing portion to create an outlet opening so that the medical media can be expelled from the flexible storage portion, wherein the improvement comprises an orifice controlling the pattern and direction of the medical media as it exits the ampoule container and wherein the orifice is spaced apart from the outlet opening.

4. The blow-molded ampoule container of claim 3, wherein the orifice is located upstream from the outlet opening.

5. The blow-molded ampoule container of claim 3, wherein the outlet opening is larger than the orifice so as not to interfere with the exit of the medical media.

6. The blow-molded ampoule container of claim 3 further comprising at least one barrier member disposed on the dispensing portion, wherein the barrier member is sized and dimensioned to limit the insertion of the dispensing member into a patient's nostril.

7. The blow-molded ampoule container of claim 3, wherein the orifice has a width between about 0.2 millimeters and about 0.8 millimeters.

8. The blow-molded ampoule container of claim 3, wherein the orifice has a width between about 0.4 millimeters and about 0.6 millimeters.

9. The blow-molded ampoule container of claim 3, wherein the orifice has a width between about 0.45 millimeters and about 0.55 millimeters.
